(19) United States
(12) Patent Application Publication
(10) Pub. No.: US 2005/0037402 A1
(43) Pub. Date: Feb. 17, 2005

(54) Device and process for direct quantitative in vitro determination of a substance that is contained in a sample

(76) Inventors: Michael Schirner, Berlin (DE); Kai Licha, Falkensee (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/887,228

(22) Filed: Jul. 9, 2004

(30) Foreign Application Priority Data
Jul. 9, 2003 (DE) 103 31 093.2

(51) Int. Cl. C12Q 1/68; G01N 33/53

(52) U.S. Cl. 435/6; 422/82.05; 436/172; 435/7.1

(57) ABSTRACT

This invention relates to a device and a process for direct quantitative in vitro determination of a substance that is contained in a sample. The device according to the invention comprises agents, immobilized on a surface, for detecting substances, a free substance-emitter conjugate, and agents, immobilized on a surface, for detecting the emitter. The emitter of the device that is used comprises a portion that reacts with a change in the emission properties in an interaction with the agent for detecting the emitter. By means of the device according to the invention, substances that are selected from antigens, such as proteins, peptides, nucleic acids, oligonucleotides, blood components, serum components, lipids, pharmaceutical agents and compounds of low molecular weight or, in another design, substance-detecting agents, such as, for example, antibodies or fragments thereof, can be determined directly quantitatively, in, e.g., whole-blood samples.

The entire disclosures of all applications, patents and publications, cited herein and of corresponding German application No. DE 103 31 093.2, filed Jul. 9, 2003, and U.S. Provisional Application Ser. No. 60/478,262, filed Jul. 16, 2003, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

This invention relates to a device and a process for direct quantitative in vitro determination of a substance that is contained in a sample. The device according to the invention comprises agents, immobilized on a surface, for detecting substances, a free substance-emitter conjugate, and agents, immobilized on a surface, for detecting the emitter. The emitter of the device that is used comprises a portion that reacts with a change in the emission properties in an interaction with the agent for detecting the emitter. By means of the device according to the invention, substances that are selected from antigens, such as proteins, peptides, nucleic acids, oligonucleotides, blood components, serum components, lipids, pharmaceutical agents and compounds of low molecular weight or antibodies or fragments thereof, can be determined directly quantitatively in, e.g., whole-blood samples.

### BACKGROUND OF THE INVENTION

For the diagnostic detection of substances and determination of concentration thereof, in many cases, in-vitro diagnostic measuring processes are now used that are based on biological molecules, such as, e.g., peptides, proteins, antibodies or oligonucleotides, which have a high affinity for a substance to be determined. Preferably used for this purpose are proteins and peptides, and especially preferably used are antibodies and antibody fragments.

In this case, the anti-substance antibodies that are used fulfill different purposes. On the one hand, they are used for separating the substance to be determined from the sample, but on the other hand, they also meet the object of locating or positioning different signal transmitters that are used on the substance to be examined. To detect, e.g., an antibody in a sample, primarily optical and radioactive measuring processes have been established, but also acoustic (see, e.g., Cooper, M. A. et al. Direct and Sensitive Detection of a Human Virus by Rupture Event Scanning. Nat Biotechnol. 2001 Separation; 19(9): 833-7) and magnetic measuring processes are known. The optical measuring processes have gained the maximum distribution [Nakamura, R. M., Dito, W. R., Tucker, E. S. (Eds.). Immunoassays: Clinical Laboratory Techniques for the 1980s. A. R. Liss, New York. Edwards, R. (ed.). Immunoassays: Essential Data, 1996, Wiley Europe].

Antibodies and peptides that are directed against molecules of low molecular weight are already known. These also include antibodies and peptides against dye molecules [Simeonov, A. et al., Science 2000, 290, 307-313; Watt, R. M. et al., Immunochemistry 1977, 14, 533-541; Rozinov, M. N. et al., Chem. Biol. 1998, 5, 713-728]. In addition, antibodies against various dyes are already commercially available, e.g., against fluorescein, tetramethylrhodamine, Texas Red, Alexa fluorine 488, BODIPY FL, Lucifer Yellow and Cascade Blue, Oregon Green (Molecular Probes Company, Inc., USA). These are, however, polyclonal IgG antibodies for bioanalytical purposes, which have partially uncontrollable cross reactivities and are not produced from a strict selection process.

Certain in vitro diagnostic processes, such as, e.g., the electrochemiluminescence, are based on the combination of various antibodies against the substance to be determined, whereby one antibody is used for the separation of the substance to be determined from the study sample, and the other antibody carries the diagnostically detected signal molecule. In the case of the diagnostic process of electrochemoluminescence, the labeled antibody is optically detected [Grayeski, M. L., Anal. Chem. 1987, 59, 1243].

In addition to the electroluminescence, the light-induced phosphorescence and the fluorescence can also be used as an optical property of molecules for diagnostic measuring processes. Compared to electroluminescence and phosphorescence, in particular fluorescence as an optical property of molecules offers the advantage of high detection sensitivity and a high linearity of the measuring signal over a large dynamic range.

To detect the fluorescence of a fluorophore, various measuring processes were developed that use different principles within the fluorescence processes. Established measuring processes use, e.g., the weakening of polarized light (fluorescence polarization=FP), the measurement of the photon service life (fluorescence service life measurement—FLM), the bleaching properties (fluorescence photobleaching recovery—FPR) and the energy transfer between various fluorophores (fluorescence-resonance-energy transfer—FRET) [Williams, A. T., et al., Methods Immunol. Anal. 1993, 1, 466; Youn, H. J. et al., Anal. Biochem. 1995 Oswald, B. et al., Anal. Biochem. 2000, 280, 272; Szollosi, J. et al., Cytometry 1998, 34, 159].

Other detection processes are based on a change in the polarization plane or the detection of phosphorescence.

In the majority of the already available measuring processes, the anti-substance antibody is labeled with a fluorophore. This labeling is carried out by specific and unspecific chemical coupling. The labeled antibody is added in excess to the study sample. This is necessary to bind all substance molecules that are to be examined. In the measurement of the fluorescence intensity as the most sensitive measuring parameter of fluorophores, it must be considered that the unbonded, labeled anti-substance antibody also delivers a fluorescence signal. For this reason, a separation of the anti-substance antibody that is specifically bonded to the substance from the unbonded portion is necessary.

In addition, this process generally uses as a basis that one anti-substance antibody uses the separation of the substance that is to be examined and the second anti-substance antibody, which detects the study substance at another binding site, is labeled with a signaling molecule. In this way, a distortion of the measuring result by the unbonded, but signaling antibody can be avoided. This procedure, however, is associated with an elevated methodical and technical expense and higher costs, produced by the separation step. The high technical expense, which prevents establishing this process for high-speed diagnosis, has proven especially disadvantageous, however.

Newer fluorescence-based measuring processes, such as, e.g., the fluorescence polarization and the fluorescence-resonance energy transfer (FRET), are methods that make it possible to determine the content of specially bonded anti-substance antibodies without separating the portion of the unbonded, fluorophore-labeled antibodies.

In this respect, the measurement of the content of a substance to be determined of unknown concentration is made possible in one step. Both methods mentioned here, however, also have decisive drawbacks, which prevent a wide distribution in in-vitro diagnosis. The fluorescence polarization thus has high detection limits in comparison to the measurement of fluorescence intensity and is thus not able to determine very small amounts of an unknown substance. Another drawback is that the measuring process cannot be used in a whole-blood sample or serum sample without being subject to noise sources, since the polarization light is influenced by a considerable number of proteins and thus it is not possible to transilluminate a blood sample without error.

FRET is also a process that can be used for detecting specifically bonded, fluorophore-labeled antibodies without separating steps, although in contrast to fluorescence polarization, the fluorescence intensity is detected as a measuring signal; the use of a whole-blood sample or serum sample is also not possible. The cause for this lies in the strong absorption and autofluorescence of the measuring sample in the wavelengths, which are currently used for the fluorescence-resonance-energy-transfer-measuring process (visible spectral range up to about 550 nm) [Mathis, G., J. Clin. Ligand Assay, 1997, 20, 141; Mathis, G., Clin. Chem. 1995, 41, 1391; Mathis, G., et. al., Clin. Chem. 1993, 39, 1251; Clarke, E. E., et. al., J. Neuroscience Methods 2000, 102, 61; Leblanc, V., et. al., Anal. Biochem. 2002, 308, 247].

It is thus the object of this invention to provide a device and an optical measuring process for the detecting reagents that are necessary for this purpose to determine the content of an unknown substance without separating steps, which can also be used, i.a., for the quantitative determination in a whole-blood sample.

This object is achieved according to the invention by the measuring process according to the invention and the provision of a device for carrying out the measuring process according to the invention according to independent claims 1, 18 and 19. Suitable configurations are cited in the dependent claims.

According to a first aspect of this invention, a device (1) for direct quantitative in vitro determination of a substance (3) that is contained in a sample is thus available, comprising a) agents that are immobilized on a surface (6) for detecting substances (5), b) free substance-emitter conjugate (2) and c) agents that are immobilized on a surface to detect emitter (4), whereby the emitter that is used comprises a portion that reacts with a change in the emission properties in an interaction with the agent for detecting emitter (4). The device according to the invention preferably comprises in addition d) agents for measuring the change in the emission properties of the emitter for direct quantitative in vitro determination of a substance that is contained in a sample.

Preferred is a device according to the invention, whereby the substance is selected from antigens, such as proteins, peptides, nucleic acids, oligonucleotides, blood components, serum components, lipids, pharmaceutical agents and compounds of low molecular weight, especially sugars, dyes or other compounds with a molecular weight of under 500 Dalton.

Further preferred is a device according to the invention, whereby the substance is an antibody or antibody fragment. In this case, a device (10) according to the invention is used for direct quantitative in vitro determination of an antibody or antibody fragment (13) that is contained in a sample, comprising, a) antigen (15) that is immobilized on a surface (16), b) free antibody (or antibody fragment)-emitter conjugate (12), and c) agents immobilized on a surface for detecting emitter (14), whereby the emitter that is used comprises a portion that reacts with a change in the emission properties in an interaction with the agent for detecting the emitter. This device according to the invention also most preferably comprises agents for measuring the change in the emission properties of the emitter for direct quantitative in vitro determination of an antibody or antibody fragment that is contained in a sample.

Further preferred is a device according to the invention, whereby the change in the emission properties of the portion of the emitter is selected from a change in the polarization plane, the fluorescence intensity, the phosphorescence intensity, the fluorescence service life and a bathochromic shift of the absorption maximum and/or the fluorescence maximum. The invention is not limited to these special phenomena, however the term “change in the emission properties” within the scope of this invention is to comprise all physical phenomena or effects in which the high-energy radiation that occurs in the emitter is altered in its property and in this case this change is quantitatively dependent on the binding/non-binding of the substance-emitter conjugate with its emitter-binding partner and/or the substance. In an embodiment of the device according to the invention, the substance is, for example, a peptide, protein, oligonucleotide and in particular an antibody or an antibody fragment. Within the scope of this invention, the antibody fragments are fragments that comprise at least the antigen-binding areas that contain the so-called “complementarity-determining regions” (“CDRs”). In this case, the antigen-binding areas most preferably comprise the completely variable chains VH and VL.

In an especially preferred aspect of the device according to this invention, the antibody or the antibody fragment is selected from polyclonal or monoclonal antibodies, humanized antibodies, Fab fragments, in particular monomeric Fab fragments, scFv fragments, synthetic and recombinant antibodies, scTCR chains and mixtures thereof.

The anti-substance antibodies or the anti-substance antibody fragment of a device of this invention optimally has a higher antigen binding affinity for the emitter than the anti-emitter antibody or the anti-emitter-antibody fragment. This affinity is selected according to the invention such that the anti-substance antibody or the anti-substance-antibody fragment has an at least 2× higher antigen-binding affinity for the emitter than the anti-emitter antibody or the anti-emitter-antibody fragment. Further preferred is that the anti-substance-antibody or the anti-substance-antibody fragment has an at least 10× higher antigen-binding affinity for the emitter than the anti-emitter-antibody or the anti-emitter-antibody fragment. Thus, a binding affinity of the antibody of less than 50 nm is preferred and less than 10 nm is more preferred. By means of the selection of the affinities, the sensitivity of the test can be optimally selected. To this end, it is advantageous that the optimal setting can be determined directly by means of conventional test series without the requirement of expensive separating steps. The same applies for the second embodiment of the test of the invention, in which an adjustment is then carried out by means of the amount of the components.

Thus, in the device according to the invention, the anti-emitter-antibody that is immobilized on the surface can be present in molar excess compared to the anti-substance antibody or compared to the immobilized antigen, whereby the ratio is preferably 1:2 to 1:50.

Another aspect of this invention relates to a device according to the invention, whereby the emitter comprises a dye that has at least one absorption maximum and/or fluorescence maximum within the spectral range of 700 to 1000 nm, preferably at least one absorption maximum and fluorescence maximum within the spectral range of 750 to 900 nm. The bathochromic shift of the dye is selected such that the shift of the absorption and/or fluorescence maximum takes place at higher wavelengths after interaction with the agent to detect the emitter by a value of greater than 15 nm, preferably greater than 25 nm, and most preferably by approximately 30 nm. In this case, a shift does not necessarily have to be considered as one that is a property of the dye. Usually, the shift would be measured as a change in one of the emission values matched to the dye, thus at a certain singular wavelength. For this purpose, the device according to the invention is preferably provided with, e.g., suitable optical agents for measurement, which are known to one skilled in the art. This also applies for the measurement of the change in the polarization plane, the fluorescence intensity, the phosphorescence intensity, the fluorescence service life and a bathochromic shift of the absorption maximum and/or the fluorescence maximum.

For the device according to the invention, it is preferred that the emitter that is used comprise a dye that is selected from the group of polymethine dyes, such as dicarbocyanine, tricarbocyanine, indotricarbocyanine, merocyanine, styrene, squarilium and oxonol dyes and rhodamine dyes, phenoxazine or phenothiazine dyes. In general, the emitter of the substance-emitter conjugate of the device according to the invention can comprise a cyanine dye of general formula (I) 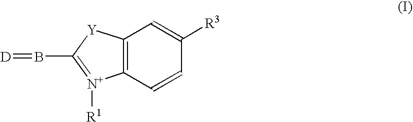
in which D stands for a radical (II) or (III) 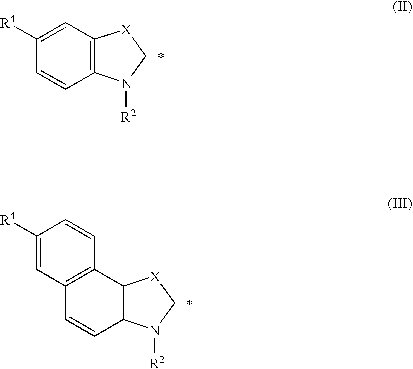
whereby the position that is labeled with the star means the point of linkage with radical B and can stand for the group (IV), (V), (VI), (VII) or (VIII) 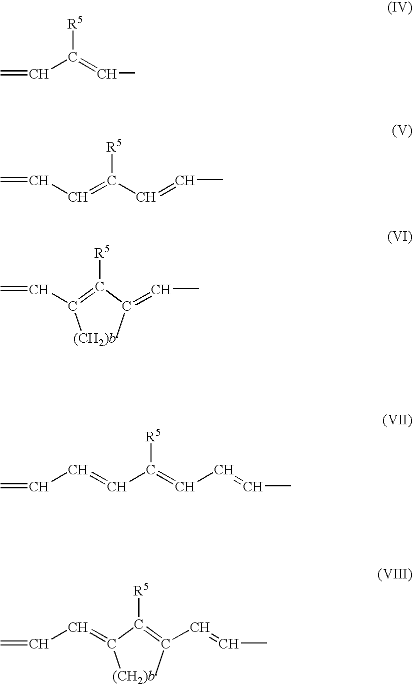
in which R¹ and R², independently of one another, represent a C₁-C₄-sulfoalkyl chain, a saturated or unsaturated, branched or straight-chain C₁-C₅₀-alkyl chain, which optionally is interrupted by 0 to 15 oxygen atoms and/or by 0 to 3 carbonyl groups and/or can be substituted with 0 to 5 hydroxy groups; R³ and R⁴, independently of one another, stand for the group —COOE¹, —CONE¹E², —NHCOE¹, —NHCONHE¹, —NE¹E², —OE¹, —OSO₃E¹, —SO₃E¹, —SO₂NHE¹ or —E¹, whereby E¹ and E², independently of one another, represent a hydrogen atom, a C₁-C₄-sulfoalkyl chain, a saturated or unsaturated, branched or straight-chain C₁-C₅₀-alkyl chain, which optionally is interrupted by 0 to 15 oxygen atoms and/or by 0 to 3 carbonyl groups and/or is substituted with 0 to 5 hydroxy groups, R⁵ stands for a hydrogen atom, a methyl, ethyl or propyl group or a fluorine, chlorine, bromine or iodine atom, b means the number 2 or 3, and X and Y independently of each other stand for O, S, ═C(CH₃)₂ or—(CH═CH)—, as well as salts and solvates of these compounds.

It was possible to find, surprisingly enough, that after highly affine binding of an antibody to a cyanine dye with absorption and fluorescence in the near-infrared spectral range (>750 nm), a shift of the absorption maximum and fluorescence maximum by about 30 mm to higher wavelengths was carried out (bathochromic shift). With use of this principle, it is thus possible, for example, via a large concentration range, to detect directly and spectrally separately a signal from a whole-blood sample, whereby the signal behaves linearly with respect to the concentration of the substance to be determined.

Within the scope of this invention, those of general formula
S-E
are used as substance-emitter conjugates, in which S stands for a substance to be examined and E stands for an emitter that comprises a portion that reacts with a change in the emission properties in an interaction with the agents for detecting the emitter. As structural components of the conjugates according to the invention, i.a., dyes that have at least an absorption maximum and a fluorescence maximum within the spectral range of 600 to 1200 nm are suitable. In this case, dyes with at least an absorption maximum and a fluorescence maximum within the spectral range of 700 to 1000 nm are preferred. Dyes that meet these criteria are, for example, those of the following classes: polymethine dyes, such as dicarbocyanine, tricarbocyanine, merocyanine and oxonol dyes, rhodamine dyes, phenoxazine or phenothiazine dyes, tetrapyrrole dyes, especially benzoporphyrins, chlorines, bacteriochlorines, pheophorbides, bacteriopheophorbides, purpurines and phthalocyanines.

Preferred dyes are the cyanine dyes with absorption maxima between 750 and 900 nm, and with special advantage indotricarbocyanines. Structural components of the conjugates according to the invention are also the substances whose determination of concentration is to be carried out by means of the process according to the invention.

These are selected from, for example, antigens, such as proteins, peptides, nucleic acids, oligonucleotides, blood components, serum components, lipids, pharmaceutical agents and compounds of low molecular weight, especially sugars, dyes or other compounds with a molecular weight of under 500 Dalton.

Analogously to this, within the scope of this invention, as substance-detecting agent-emitter-conjugates, those of general formula
SEM-E
are used, in which SEM stands for a substance-detecting agent that is to be examined and E stands for an emitter that comprises a portion that reacts with a change in the emission properties in an interaction with the agents for detecting the emitter. As structural components of the conjugates according to the invention, i.a., dyes that have at least an absorption maximum and a fluorescence maximum within the spectral range of 600 to 1200 nm are suitable. In this case, dyes with at least an absorption maximum and a fluorescence maximum within the spectral range of 700 to 1000 nm are preferred. Dyes that meet these criteria are, for example, those of the following classes: polymethine dyes, such as dicarbocyanine, tricarbocyanine, merocyanine and oxonol dyes, rhodamine dyes, phenoxazine or phenothiazine dyes, tetrapyrrole dyes, especially benzoporphyrins, chlorines, bacteriochlorines, phenophorbides, bacteriopheophorbides, purpurines and phthalocyanines.

Preferred dyes are the cyanine dyes with absorption maxima between 750 and 900 nm, and with special advantage indotricarbocyanines. Structural components of the conjugates according to the invention are also the substance-detecting agents whose determination of concentration is to be carried out by means of the process according to the invention.

These are selected from, for example, peptides, proteins, oligonucleotides, and especially antibodies or antibody fragments.

The dyes contain structural elements, via which the covalent coupling to the substance structures or the substance-detecting structures is carried out. The latter are, e.g., linkers with carboxy groups, amino groups, and hydroxy groups. In the case of an antigen-antibody-bond, the conjugate from the substance to be examined and the emitter has a binding affinity, on the one hand, for the antibody against the substance to be examined and, on the other hand, for the antibody against the emitter portion (e.g., fluorophore). After binding the anti-fluorophore antibody to the fluorophore, a shift of the absorption and/or fluorescence maximum takes place. In this case, a shift of the absorption and fluorescence maximum preferably takes place at higher wavelengths by a value of greater than 15 nm. This value is especially preferably greater than 25 nm.

In another aspect of the device according to the invention, the agents and/or the antigen (substance) are present directly or indirectly on a surface that is statistically random or is immobilized in a targeted manner. “Indirect” immobilization is defined here as the coupling of agents via a suitable “linker.” Such linkers are extensively used, e.g., in the technology of biological “chips” and are well known to one skilled in the art. The general function of the linker is the fixing and optional exact positioning of the agents on the surface. In this case, the fixing can be carried out covalently or non-covalently. Usually, the linker is not involved in the binding between the substance to be detected and the immobilized agent. Examples of linkers extend from, e.g., PNA oligomers, silane-containing groups and succinimide groups up to groups that form peptide bonds. The “direct” immobilization on the surface is carried out without further intermediate groups, for example by an activated terminal group of the agent. Such chemically activated groups are also well known to one skilled in the art. By means of the above-mentioned immobilizing groups, the distribution of agents on the surface can take place statistically randomly or in a targeted manner. The targeted immobilization allows, for example, the introduction of various detecting agents on a surface and thus, e.g., the production of a surface that can be simultaneously suitable for several tests.

According to the invention, the device comprises a membrane, a ball (pearl or “bead”) or a solid flat surface, whereby these vehicles can consist of nylon, cellulose and derivatives thereof, resin matrix, silicon, glass, polystyrene, aluminum, steel, iron, copper, nickel, silver or gold. These are conventional materials for such surfaces, which can be produced easily.

Another aspect of this invention then relates to a process for direct quantitative in vitro determination of a substance (3) that is contained in a sample, comprising the steps of, a) making available a device (1) that comprises, i) agents immobilized on a surface (6) for detecting substances (5), ii) a free substance-emitter conjugate (2), and iii) agents that are immobilized on a surface for detecting emitter (4), whereby the emitter that is used comprises a portion that reacts with a change in the emission properties in an interaction with the agents for detecting the emitter, b) bringing into contact the device with a sample that contains a substance that is to be quantified, and c) measuring the change in the emission properties of the emitter.

Another aspect of this invention then relates to a process for direct quantitative in vitro determination of a substance-detecting agent that is present in the sample, comprising the steps of a) making available a device (10) that comprises i) substance (15) that is immobilized on a surface (16), ii) a free substance-detecting agent-emitter conjugate (12), and iii) agents that are immobilized on a surface for detecting emitter (14), whereby the emitter that is used comprises a portion that reacts with a change in the emission properties in an interaction with the agents for detecting the emitter, b) bringing into contact the device with a sample that contains a substance that is to be quantified or a substance-detecting agent, and c) measuring the change in the emission properties of the emitter. In addition, the device here also comprises agents for measuring the change in the emission properties of the emitter.

Preferred is a process according to the invention for quantitative in vitro determination of a substance that is contained in a sample, which in addition comprises d) quantification of the substance or substance-detecting agent that is contained in the sample by means of the measured change in the emission properties of the emitter.

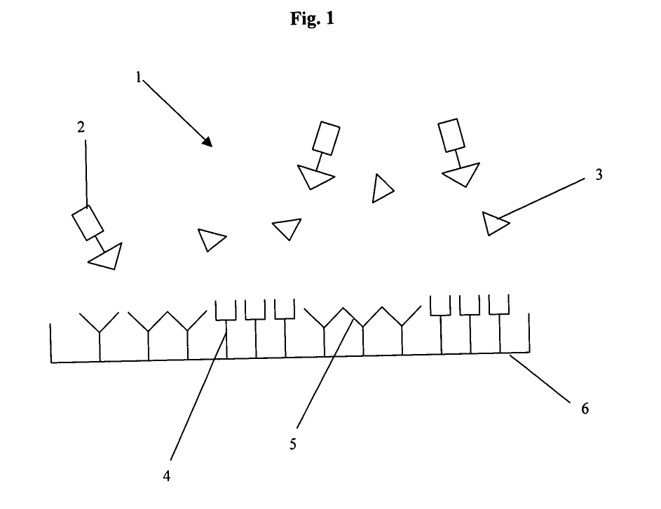

The measuring process according to the invention is based in a preferred embodiment (see FIG. 1) on the use of a conjugate (2) that consists of the substance to be determined and an emitter (especially fluorophore) in combination with two antibodies (4, 5), whereby one antibody binds the substance to be determined as an antigen (anti-substance antibody, 5) and the other antibody binds the emitter as an antigen (anti-emitter-antibody, 4). Conjugate (2) according to the invention from the substance to be determined and the emitter concurs with free substance (3) that is to be determined in the sample for the binding sites of substance-binding antibody (5). The higher the content of substance (3) to be determined in the study sample is, the greater conjugate (2) is displaced from the antigen-binding site of the anti-substance-antibody and binds to anti-emitter antibody (4) in increasing concentration (see FIG. 1). Anti-emitter antibody (5) is distinguished in that the spectral properties of the emitter are characteristically changed by the binding of the emitter in the antigen-binding pocket of the antibody. In this respect, it is possible to determine separately the portion of substance-emitter conjugate (2), which is bonded to anti-emitter-antibody (4), with use of the spectral difference from the portion that is bonded to anti-substance antibody (5).

Substance-emitter conjugate (2) competes with substance (3) to be examined for the binding sites to the antibody, which is directed against the substance that is to be examined (5). Increasing concentrations of substance (3) that is to be examined in the study sample displace the binding of the conjugate in the direction of antibody (4), which is directed against the emitter.

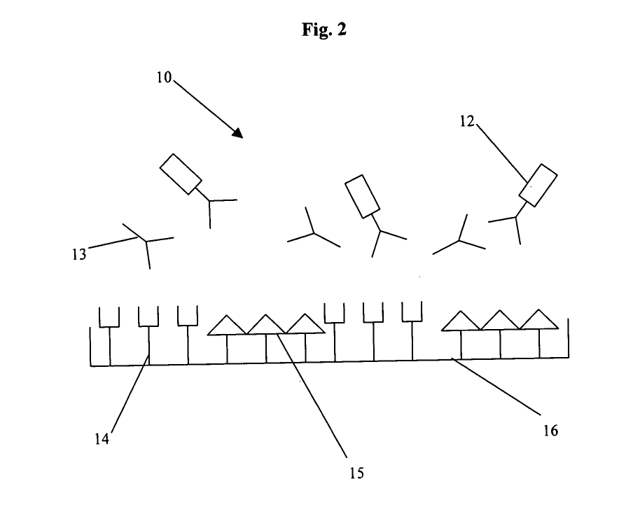

In another preferred embodiment (see FIG. 2), the measuring process according to the invention is based on the use of a conjugate (12) that consists of a specific substance-detecting agent, e.g., an antibody, and an emitter (especially fluorophore) in combination with an antibody and antigen (13, 14), whereby the antigen binds the specific substance (e.g., antibodies (anti-substance antibody, 13) and the antibody binds the emitter as an antigen (anti-emitter antibody, 14). The conjugate that consists of the substance-detecting agent and the emitter concurs with free antibody (13) that is to be determined in the sample for immobilized substance (15). The higher the content of the substance-detecting agent to be determined, e.g., antibody (13), in the study sample is, the greater the conjugate is displaced from the antigen-binding site of anti-substance-antibody (13) and binds to anti-emitter antibody (14) in increasing concentration (FIG. 2). Anti-emitter antibody (14) is distinguished in that the spectral properties of the emitter are characteristically changed by the binding of the emitter in the antigen-binding pocket of the antibody. In this respect, it is possible to determine separately the portion of the substance-detecting agent (e.g., antibody)-emitter conjugate (12), which is bonded to anti-emitter-antibody (14), with use of the spectral difference from the portion that is bonded to substance (15).

Substance-detecting-agent-emitter conjugate (12) competes with substance-detecting agent (13) to be examined for the binding sites to substance (5). Increasing concentrations of substance-detecting agent (13) to be examined in the study sample shift the binding of the conjugate in the direction of antibody (14), which is directed against the emitter.

Preferred according to the invention is a process whereby the substance that is to be determined is selected from antigens, such as proteins, peptides, nucleic acids, oligonucleotides, blood components, serum components, lipids, pharmaceutical agents and compounds of lower molecular weight, especially sugars, dyes or other compounds with a molecular weight of less than 500 Dalton. The substance-detecting agents are selected, for example, from peptides, proteins, oligonucleotides and in particular antibodies or antibody fragments.

Further preferred is a process according to the invention, whereby the change in the emission properties of the portion of the emitter is selected from a change of the polarization plane, the fluorescence intensity, the phosphorescence intensity, the fluorescence service life and a bathochromic shift of the absorption maximum and/or the fluorescence maximum. The invention, however, is not limited to these special phenomena: the term “change in the emission properties” within the scope of this invention is to comprise all physical phenomena or effects in which the high-energy radiation that occurs in the emitter is altered in its property and in this case this change is quantitatively dependent on the binding/non-binding of the substance-emitter conjugate or substance-detecting agent-emitter conjugate with its emitter-binding partner and the substance. In an embodiment of the device according to the invention, the substance is, for example, a peptide, protein, oligonucleotide and in particular an antibody or an antibody fragment.

In the case of an optical measurement, the latter can be carried out in a different way and is directed mainly according to the type of characteristic change in the spectral property of the emitter (e.g., fluorophore). Generally preferred is a detection of the shift of the absorption wavelength and emission wavelength or the measurement of the absorption and/or fluorescence intensity at a wavelength that for the most part detects the portion of the emitter that is bonded to the antibody. Depending on the change in the spectral properties of the antibody-bonded emitter, other properties, such as, e.g., the photon service life, the polarization, and the bleaching behavior can also be used for optical measurement.

The special advantage of fluorophores in the spectral range of near-infrared light lies in the low rate of shadowing by components of the blood. In this respect, deep penetration is made possible without the signal to be detected being relatively changed to any major extent.

Preferred is a process according to the invention whereby as the substance-detecting agents, peptides, proteins and especially antibodies or antibody fragments are brought into contact with the sample. In an especially preferred aspect of the process according to this invention, the antibody or the antibody fragment is selected from polyclonal or monoclonal antibodies, humanized antibodies, Fab fragments, especially monomeric Fab fragments, scFv fragments, synthetic and recombinant antibodies, scTCR chains and mixtures thereof.

The antibodies that are used can be bivalent whole immunoglobulins, but monomeric Fab fragments are preferably used in the test system. After free binding sites to the solid phase are blocked, the system is calibrated at constant substance-fluorophore concentration with saturating concentration and increasing substance concentration. The sample to be determined is used for measuring either in diluted form but preferably in undiluted form.

Antibodies that are directed against substances to be examined (anti-substance antibodies) are already known. According to the invention, anti-substance antibodies with a high affinity for the substance are preferred. Simeonov, A. et al. describe in Science 200, 290, 307-313 antibodies against stilbene (“blue-fluorescent antibodies”). The antibodies catalyze specific photochemical isomerization processes and result in red-shifted absorption and fluorescence maxima in the UV-VIS spectral range (absorption shift maximum 12 nm, fluorescence shift 22 nm). Simeonov, A. et al. provide no reference whatsoever to a red shift while preserving the fluorescence quantum yield in the case of cyanine dyes in a wavelength range of 600-1200 nm.

Watt, R. M. et al. (Immunochemistry 1977, 14, 533-541) describe the spectral properties of the already known anti-fluorescein-antibody construct. After binding the fluorescein, the antibody produces a shift of the absorption and fluorescence maximum in the visible spectral range, but only by 12 nm or 5 nm. In addition, a strong reduction of the fluorescence quantum yield (by about 90%) is carried out. The red shifts of the antibody-dye-constructs used according to the invention are >15 nm in the NIR spectral range while preserving the fluorescence quantum yield.

Rozinov, M. N. et al. (Chem. Biol. 1998, 5, 713-728) finally describe the selection of 12-mer peptides from phage libraries, which bind the dyes Texas Red, Rhodamine Red, Oregon Green 514 and fluorescein. For Texas Red, a red shift of the absorption and fluorescence was observed, but only by 2.8 nm or 1.4 nm. Rozinov et al. do not propose, however, that antibodies against cyanine dyes lead to larger shifts while preserving the fluorescence quantum yield and therefore are suitable for the process according to the invention.

Moreover, antibodies against fluorophores, which are able to change their spectral properties in the UV range after binding the fluorophore, are already known to one skilled in the art. By binding a fluorophore in the antigen binding pocket of an antibody, primarily the fluorescence intensity, the absorption maximum, the emission maximum, and the photon service life can be changed [Simeonov, A., et al., Science (2000) 307-313]. These known antibodies are directed against emitters (fluorophores), however, which have their absorption and fluorescence emission in the visible and UV range of the light.

The anti-substance antibody or the anti-substance-antibody fragment in the process of this invention optimally exhibits a higher antigen-binding affinity for the emitter than the anti-emitter antibody or the anti-emitter-antibody fragment. This affinity is selected according to the invention such that the anti-substance antibody or the anti-substance antibody fragment has an at least 2× higher antigen binding affinity for the emitter than the anti-emitter antibody or the anti-emitter-antibody fragment. It is further preferred that the anti-substance antibody or the anti-substance-antibody fragment exhibit an at least 10× higher antigen binding affinity for the emitter than the anti-emitter antibody or the anti-emitter-antibody fragment. Thus, a binding affinity of the antibody of less than 50 nm is preferred, and less than 10 nm is further preferred. By means of the selection of affinities, the sensitivity of the test can be optimally selected. For this purpose, it is advantageous that the optimal setting can be determined directly by conventional test series, without the requirement of expensive separating steps. The same applies for the second embodiment of the test of the invention, in which then an adjustment is carried out by means of the amount of components.

The measuring process according to the invention thus preferably uses anti-substance antibodies that have a higher antigen-binding affinity than the anti-fluorophore antibodies. Preferred are anti-substance antibodies with an at least 2× higher binding affinity compared to the anti-fluorophore antibodies. Especially preferred are anti-substance antibodies with a more than 10× higher binding affinity. The anti-fluorophore antibodies are preferably directed against fluorophores that absorb and emit in the spectral range of near-infrared light and their spectral properties are changed by the binding to the antibodies in the sense that the emission signal of the antibody-bonded portion of the fluorophore can be spectrally detected separately from the free portion of the fluorophore.

Thus, in the process according to the invention, the anti-emitter-antibody that is immobilized on the surface can be present in molar excess compared to the anti-substance-antibody or compared to the immobilized antigen, whereby the ratio is preferably from 1:2 to 1:50.

Another aspect of this invention relates to the use of a device according to the invention for in vitro diagnosis. Still another aspect relates to the use of a substance-emitter conjugate or emitter-detecting agent, especially a substance-fluorophore conjugate or anti-fluorophore antibody for in vitro diagnosis.

For this purpose, the device according to the invention can also be present in a diagnostic kit, in which the components of the device, optionally together with other adjuvants, are available together or in separate containers. Another possibility consists in a first kit that makes available the basic elements of the device of the invention (e.g., suitable surface and antibodies and/or substance coupled thereto), which then together with the contents of a second kit (containing a substance for calibration and/or other antibodies) is “specialized” for the respective application. Such a second kit could contain, for example, a specific substance-emitter conjugate. In addition, all of these kits can contain special instructions and documents (e.g., calibration curves, directions for quantification, etc.).

The invention is now to be described in more detail below based on examples with reference to the attached figures, without, however, being limited thereto. Here:

FIG. 1: shows a first embodiment of the device of the invention,

FIG. 2: shows a second embodiment of the device of the invention, and

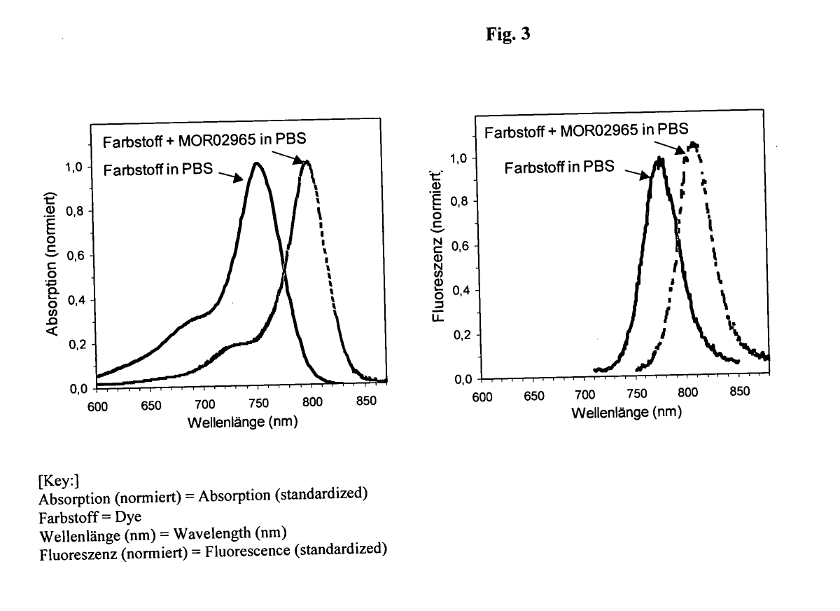

FIG. 3: shows the absorption spectrum (left) and fluorescence spectrum of the dye with and without the presence of antibody MOR02965 in PBS from Example 2.

### EXAMPLES

### Example 1

### Selection, Production and Characterization of Emitter-Binding Antibodies: Selection of HuCAL GOLD Antibody Fragments against the Cyanine Dye Fuji 6-4 (ZK203468) [Trisodium-3,3-dimethyl-2-{4-methyl-7-[3,3-dimethyl-5-sulfonato-1-(2-sulfonatoethyl)-3H-indolium-2-yl]hepta-2,4,6-trien-1-ylidene}-1-(2-sulfonatoethyl)-2,3-dihydro-1H-indole-5-sulfonate, Inner Salt]

HuCAL GOLD Antibody Library:

Antibody library HuCAL GOLD. HuCAL GOLD is a fully synthetic, modular human antibody library in the Fab antibody fragment format. HuCAL GOLD is based on the HuCAL-consensus-antibody genes that were described for the HuCAL-scFv1 library (WO 97/08320; Knappik, (2000), J. Mol. Biol. 296, 57-86; Krebs et al. J Immunol Methods. 2001 Aug. 1; 254(1-2):67-84). In HuCAL GOLD, all six CDR areas are diversified by the use of so-called trinucleotide mutagenesis (Virnekäs et al. (1994) Nucleic Acids Res. 1994 Dec. 25; 22(25):5600-7) corresponding to the composition of these areas in human antibodies, while in earlier HuCAL libraries (HuCAL-scFv1 and HuCAL-Fab1, only the CDR3-areas in VH and VL would be diversified corresponding to the natural composition (see Knappik et al., 2000). Moreover, an amended screening process, the so-called CysDisplay (WO 01/05950), is also found in HuCAL GOLD.
Vλ Positions 1 and 2. The original HuCAL master genes were constructed with their authentic N-termini: VLλ1: QS (CAGAGC), VLλ2: QS (CAGAGC), and VLλ3: SY (AGCTAT). These sequences are found in WO 97/08320. In the production of the HuCAL-scFv1-library, these two amino acid radicals were changed in “DI” to facilitate the cloning (EcoRI site). These radicals were preserved in the production of HuCAL-Fab1 and HuCAL GOLD. All HuCAL libraries therefore contain VLλ genes with the EcoRV interface GATATC (DI) at the 5′-end. All HuCAL kappa genes (master genes and all genes in the libraries) in any case contain DI at the 5′-end, since these represent the authentic N-termini (WO 97/08320).
VH Position 1. The original HuCAL-master genes were produced with their authentic N-termini: VH1A, VH1B, VH2, VH4, and VH6 with Q (=CAG) as a first amino acid radical and VH3 as well as VH5 with E (=GAA). The corresponding sequences are found in WO 97/08320. In the cloning of HuCAL-Fab1 as well as the HuCAL GOLD library, the amino acid Q (CAG) was incorporated in all VH genes at this position 1.
   Phagemid Production

Large amounts of phagemids were produced and concentrated by infection of E. coli TOP10F′ cells from the HuCAL GOLD antibody library or from the maturation libraries by means of helper phages. To this end, the HuCAL GOLD or the maturation libraries (in the TOP10F′ cells) were cultivated in 2×YT medium with 34 μg/ml of chloramphenicol/10 μg/ml of tetracycline/1% glucose at 37° C. up to an OD₆₀₀ of 0.5. Then, the infection was carried out with VCSM13 helper phages at 37° C. The infected cells were pelletized and resuspended in 2×YT/34 μg/ml of chloramphenicol/10 μg/ml of tetracycline/50 μg/ml of kanamycin/0.25 mmol of IPTG and cultivated overnight at 22° C. The phages were precipitated 2× with PEG from the supernatant and harvested by centrifuging (Ausubel (1998) Current Protocols in Molecular Biology. John Wiley Sons, Inc., New York, USA). The phages were resuspended in PBS/20% glycerol and stored at −80° C.

The phagemid amplification between the individual selection rounds was carried out as follows: log-phase E. coli TG1 cells were infected the with selected phages and flattened out on LB-agar plates with 1% glucose/34 μg/ml of chloramphenicol. After incubation overnight, the bacteria colonies were scraped off, newly cultivated and infected with VCSM13 helper phages.

Primary Selection of Antibodies Against the Dye Fuji 6-4 (ZK203468)

The purified and concentrated phagemids of the HuCAL GOLD antibody library were used in a standard selection process. As antigens, BSA- or transferrin-coupled ZK203468 were used alternately. The antigens were taken up in PBS and applied at concentrations of 50 μg/ml on Maxisorp™ microtiter plates F96 (Nunc). The maxisorp plates were incubated overnight at 4° C. (“coating”). After the maxisorp plates were blocked with 5% milk powder in PBS, about 2E+13 HuCAL GOLD phages were added to the antigen-loaded, blocked-off spots and incubated there overnight or for two hours at room temperature. After several washing steps, which became more stringent with progressive selection rounds, bonded phages were eluted with 20 mmol of DTT or 100 μmol of unconjugated ZK203468. Altogether, three successive selection rounds were carried out, whereby the phage amplification was carried out between the selection rounds, as described above.

Sub-Cloning of Selected Fab Fragments for Expression

After the antibody selection that comprises three rounds, the Fab-coding inserts of the isolated HuCAL clones were subcloned in the expression vector pMORPHX9_MS to facilitate the subsequent expression of the Fab fragments. To this end, the purified plasmid-DNA of the selected HuCAL Fab clones was digested with the restriction enzymes XbaI and EcoRI. The Fab-coding insert was purified and ligated in the correspondingly digested vector pMORPHX9_MS. This cloning step results in the Fab-expressing vector pMORPHX9_Fab_MS. Fab fragments, which are expressed by this vector, carry two C-terminal tags (Myc tag and Strep tag II) for purification and detection.

Screening and Characterization of ZK203468-Binding Fab Fragments

Several thousand clones were isolated after the selection and sub-cloning and tested by means of ELISA in 384-well format for specific detection of the antigens ZK203468-BSA and—transferrin used in panning. Clones identified in this connection were studied in an inhibition-ELISA for efficient binding of the unconjugated dye. This resulted in the parenteral Fab fragments MOR02628 (protein sequences SEQ-ID NO: 1 (VH-CH) and SEQ-ID NO: 2 (VL-CL); DNA sequences SEQ-ID NO: 3 (VH-CH) and SEQ-ID NO: 4 (VL-CL)), MOR02965 (protein sequences SEQ-ID NO: 5 (VH-CH) and SEQ-ID NO: 6 (VL-CL); DNA-sequences SEQ-ID NO: 7 (VH-CH) and SEQ-ID NO: 8 (VL-CL)) and MOR02977 (protein sequences SEQ-ID NO: 9 (VH-CH) and SEQ-ID NO: 10 (VL-CL); DNA sequences SEQ-ID NO: 11 (VH-CH) and SEQ-ID NO: 12 (VL-CL)), that bind efficiently to the non-conjugated dye ZK203468.

### Example 2

### Photophysical Characterization of Dye-Antibody Complexes and Determination of Spectral Shifts/Fluorescence Quantum Yields

Dye-antibody complexes based on antibodies with binding to the indotricarbocyanine dye trisodium-3,3-dimethyl-2-{4-methyl-7-[3,3-dimethyl-5-sulfonato-1-(2-sulfonatoethyl)-3H-indolium-2-yl]hepta-2,4,6-trien-1-ylidene}-1-(2-sulfonatoethyl)-2,3-dihydro-1H-indole-5-sulfonate, inner salt, were examined (see Example 1). Solutions of the concentration of 1 μmol/l of the above-mentioned dye and 2.4 μmol/l of the respective antibody in PBS were produced and incubated for 2 hours at room temperature. The absorption maxima were determined with a spectral photometer (Perkin-Elmer, Lambda2). The fluorescence maxima and fluorescence quantum yields were determined with a SPEX fluorolog (wavelength-dependent sensitivity calibrated by lamp and detector) relative to indocyanine green (Q=0.13 in DMSO, J Chem Eng Data 1977, 22, 379, Bioconjugate Chem 2001, 12, 44). From the absorption and fluorescence maxima, the spectral shifts were calculated relative to the maxima of a solution of the above-mentioned dye without antibodies in PBS (1 μmol/l) (absorption max. 754 nm, fluorescence max. 783 nm, fluorescence quantum yield 10%).

The results are summarized in Table 1 below:

TABLE 1

| | | Absorption | Fluorescence | Absorption | Fluorescence | Fluorescence |
|---|---|---|---|---|---|---|
| | Parenteral | Maximum | Maximum | Shift | Shift | Quantum |
| Antibody | Antibody | (nm) | (nm) | (nm) | (nm) | Yield (%) |
| Free Dye | | 754 | 783 | — | — | 10.0 |
| MOR02628 | | 768 | 795 | 14 | 12 | 8.1 |
| MOR02965 | — | 799 | 815 | 45 | 32 | 13.0 |
| MOR02977 | — | 773 | 788 | 19 | 5 | 24.5 |

### Example 3

### Synthesis of the Conjugate from Substance-Detecting Means and Emitters: Anti-ED-B-Fibronectin Antibodies/Indotricarbocyanine Conjugate

Antibodies against the extremely pure recombinant ED-B domains of fetal fibronectin are preferably used as scFv, Fab, (Fab)₂ or as whole-IgG. In this example, a Fab with a C-terminal cystein-tag was used and covalently conjugated with a linker-modified derivative of the indotricarbocyanine dye trisodium-3,3-dimethyl-2-{4-methyl-7-[3,3-dimethyl-5-sulfonato-1-(2-sulfonatoethyl)-3H-indolium-2-yl]hepta-2,4,6-trien-1-ylidene}-1-(2-sulfonatoethyl)-2,3-dihydro-1H-indole-5-sulfonate, inner salt. The following operating steps are carried out:

### Synthesis of Trisodium 3,3-dimethyl-2-{7-[3,3-dimethyl-5-sulfonato-1-(2-sulfonatoethyl)-3H-indolium-2-yl]-4-(5-{[2-(2,5-dioxo-2,5-dihydro-1H-pyrrol-1-yl)ethyl]carbamoyl}-3-oxa-pentyl)hepta-2,4,6-trien-1-ylidene}-1-(2-sulfonatoethyl)-2,3-dihydro-1H-indole-5-sulfonate, Inner Salt for Conjugation on Fab

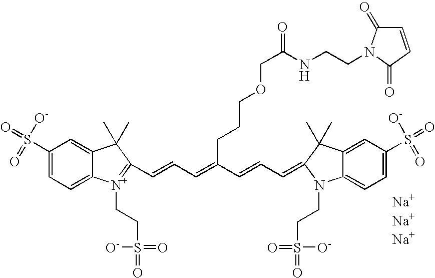 a) 3-Oxa-6-(4-Pyridinyl)hexanoic acid-tert-butyl ester

A solution of 75 g (0.4 mol) of 3-(4-pyridinyl)-1-propanol in 400 ml of toluene/50 ml of THF is mixed with 10 g of tetrabutylammonium sulfate and 350 ml of 32% sodium hydroxide solution. Then, 123 g (0.68 mol) of bromoacetic acid-tert-butyl ester is added in drops and stirred for 18 hours at room temperature. The organic phase is separated, and the aqueous phase is extracted three times with diethyl ether. The combined organic phases are washed with NaCl solution, dried on sodium sulfate and concentrated by evaporation. After chromatographic purification (silica gel; mobile solvent hexane:ethyl acetate), 56 g of product (41% of theory) is obtained as a brownish oil.

b) 3-[4-Oxa-5-(tert-butyloxycarbonyl)pentyl]glutaconaldehyde-dianilide-hydrobromide

A solution of 5.0 g (20 mmol) of 3-oxa-6-(4-pyridinyl)hexanoic acid-tert-butyl ester in 60 ml of diethyl ether is mixed with 3.7 g (40 mmol) of aniline and then mixed at 0° C. with a solution of 2.2 g (20 mmol) of bromocyanogen in 8 ml of diethyl ether. After 1 hour of stirring at 0° C., it is mixed with 50 ml of diethyl ether, and the red solid that is produced is filtered off, washed with ether and vacuum-dried. Yield: 8.5 g (85% of theory) of a violet solid.

c) Trisodium 3,3-dimethyl-2-{7-[3,3-dimethyl-5-sulfonato-1-(2-sulfonatoethyl)-3H-indolium-2-yl]-4-(6-carboxy-4-oxahexyl)hepta-2,4,6-trien-1-ylidene}-1-(2-sulfonatoethyl)-2,3-dihydro-1H-indole-5-sulfonate, inner salt

A suspension of 3.0 g (6 mmol) of 3-[2-(tert-butyloxycarbonyl)ethyl]-glutaconaldehyde-dianilide-hydrobromide (Example 10b) and 4.2 g (12 mmol) of 1-(2-sulfonatoethyl)-2,3,3-trimethyl-3H-indolenine-5-sulfonic acid (Example 1a) in 50 ml of acetic acid anhydride and 10 ml of acetic acid is mixed with 2.5 g (30 mmol) of sodium acetate and stirred for 50 minutes at 120° C. After cooling, it is mixed with diethyl ether, the precipitated solid is filtered off, it is absorptively precipitated in acetone and dried. After chromatographic purification (RP-C18 silica gel, mobile solvent water/methanol), removal of the methanol in a vacuum and freeze-drying, the title compound is obtained directly. Yield: 2.3 g (41% of theory) of a blue lyophilizate.

d) Trisodium 3,3-dimethyl-2-{7-[3,3-dimethyl-5-sulfonato-1-(2-sulfonatoethyl)-3 H-indolium-2-yl]-4-(5-{[2-(2,5-dioxo-2,5-dihydro-1H-pyrrol-1-yl)ethyl]carbamoyl}-3-oxa-pentyl)hepta-2,4,6-trien-1-ylidene}-1-(2-sulfonatoethyl)-2,3-dihydro-1H-indole-5-sulfonate, inner salt

1.0 g (1.1 mmol) of the title compound of Example Xe and 0.1 g (1.1 mmol) of triethylamine are dissolved in 15 ml of dimethylformamide, mixed at 0° C. with 0.37 g (1.1 mmol) of TBTU and stirred for 15 minutes. Then, a solution of 0.42 g (1.7 mmol) of N-(2-aminoethyl)maleimide-trifluoroacetate (Int J Pept Protein Res 1992, 40, 445) and 0.17 mg (1.7 mmol) of triethylamine in 1.0 ml of dimethylformamide is added and stirred for 1 hour at room temperature. After 30 ml of diethyl ether is added, the solid is centrifuged off, dried and purified by means of chromatography (RP-C-18 silica gel, methanol/water gradient). Yield: 0.85 g of a blue lyophilizate (73% of theory).

Synthesis of an Indotricarbocyanine-Fab Conjugate

0.3 ml of a solution of Fab antibodies in PBS (conc. 0.8 mg/ml) is mixed with 60 μl of a solution of tris(carboxyethyl)phosphine (TCEP) in PBS (2.8 mg/ml) and incubated under nitrogen for 1 hour at 25° C. Excess TCEP is separated by gel filtration on an NAP-5 column (eluant: PBS). The amount of Fab that is obtained that is determined by photometry (OD₂₈₀ₙₘ=1.4) is 230-250 μg (volumes 0.5-0.6 ml). The solution is mixed with 0.03 μmol of trisodium 3,3-dimethyl-2-{7-[3,3-dimethyl-5-sulfonato-1-(2-sulfonatoethyl)-3H-indolium-2-yl]-4-(5-{[2-(2,5-dioxo-2,5-dihydro-1H-pyrrol-1-yl)ethyl]carbamoyl}--3-oxa-pentyl)hepta-2,4,6-trien-1-ylidene}-1-(2-sulfonatoethyl)-2,3-dihydro-1H-indole-5-sulfonate, inner salt (stock solutions of 0.5 mg/ml in PBS), and incubated for 30 minutes at 25° C. The conjugate is purified by gel chromatography on a NAP-5 column (eluant: PBS/10% glycerol). The immune reactivity of the conjugate solution is determined by means of affinity chromatography (ED-B-fibronectin resin) (J Immunol Meth 1999, 231, 239) and is ˜75%.

### Example 4

### In-vitro Assay for Quantification of Embryonal Fibronectin (ED-B-Fibronectin) in Human Serum

The quantification of circulating fetal fibronectin (ED-B fibronectin) (Curr Opin Drug Discov Devel. 2002, 5, 204) is based on ELISA techniques. To this end, transparent 96-well Immunosorb-ELISA plates, but preferably white Immunosorb-ELISA plates (Nunk, Denmark) are used for chemoluminescence detection. The following operating steps are carried out:

Coating of the ELISA Plates

The extremely pure recombinant ED-B domains of the fetal fibronectin (ED-B-FN) is immobilized on the surface of the ELISA plates as an antigen together with the extremely pure anti-emitter antibodies (see Example 1), which preferably can be present as scFv, Fab, (Fab)₂ or as whole-IgG. As a coupling buffer, PBS, but preferably alkaline coupling buffer, is used. The latter is composed of a mixture of 17 ml of a 0.2 M Na₂CO₃ solution and 8 ml of an NaHCO₃ solution, which, made up to 100 ml with bidistilled water, yield the ready-to-use coupling buffer. The concentration of recombinant antigen and anti-emitter antibody is in the range of 1-10 μg/ml, whereby the optimal molar ratio of recombinant antigen and anti-emitter antibody must be determined empirically. Preferably, however, ratios in the range of 1:5-1:100 are selected. The coupling is carried out either for 2 hours at 37° C. or else at 4° C. overnight in a volume of 100 μl per spot.

Blocking of Free Binding Sites

After coupling is completed, free binding sites are blocked on the ELISA plates with PBS, which contains 2% (w/v) bovine serum albumin or gelatin, but preferably with blocking buffer with 2% (w/v) bovine albumin or gelatin. To this end, the plate is rapped after coupling to remove excess material and incubated with 200 μl of blocking buffer for 2 hours at 37° C.

Calibration

To calibrate the system, a calibration series of extremely pure ED-B-FN is pipetted into human serum. To this end, an ED-B-FN sample with a concentration of 10 μg/ml is diluted serially in 1:2-steps, whereby each sample contains a constant concentration of anti-substance antibodies, labeled with emitters, (anti-ED-B-FN Fab-indotricarbocyanine conjugate from Example 3). The latter can lie in the range of 0.1 μg/ml to 10 μg/ml corresponding to the test system.

Pipetting of the Quantitative ELISA

The blocked ELISA plate is rapped to remove excess material, and in each case 100 μl of the calibration protein series is pipetted in triplicate into the respective holes in the ELISA plate. The serum or whole-blood samples to be determined are applied in triplicate on the plate and incubated for 30 minutes at 37° C.

Measurement of the Samples

The measurement of the samples is carried out in a spectral fluorometer with two monochromators (SPEX-Fluorog, Jobin Yvon). The wavelength of the excitation light and the detection wavelength for the emitted fluorescence can be freely selected. The samples are examined in an ELISA plate module. This example is selected as excitation wavelength 790 nm. The detection of fluorescence is carried out in a bandpass of 805-860 nm. As a result, the increase of the fluorescence signal is detected in a red-shifted wavelength (cf. Figure) by increasing the portion of anti-substance emitter conjugate, which binds to immobilized anti-emitter antibodies via the dye. Typically, a sigmoid curve plot is found, whereby the linear measuring range of the calibration series is used for quantitative determination of the measuring samples.

### Example 5

### Antibodies Against ED-B-Fibronectin

Antibodies against ED-B-fibronectin were generated analogously to the procedure described in Example 1 from the HuCAL GOLD-antibody library against ED-B-fibronectin as an antigen.

Legend

(1) Device for measuring a substance in a sample
(2) Substance-emitter conjugate
(3) Substance
(4) Emitter-detecting agent
(5) Substance-detecting agent
(6) Surface
(10) Device for measuring an antibody-detecting agent in a sample
(12) Antibody-emitter conjugate
(13) Antibody
(14) Emitter-detecting agent
(15) Immobilized antigen
(16) Surface

1. Device for direct quantitative in vitro determination of a substance that is contained in a sample, comprising
a) agents that are immobilized on a surface for detecting substances,
b) a free substance-emitter conjugate, and
c) agents that are immobilized on a surface for detecting the emitter, whereby the emitter that is used comprises a portion that reacts with a change in the emission properties in an interaction with the agent for detecting the emitter.

2. Device for direct quantitative in vitro determination of a substance that is contained in a sample according to claim 1, further comprising
d) agents for measuring the change in the emission properties of the emitter.

3. Device according to claim 1, whereby the substance is selected from antigens, such as proteins, peptides, nucleic acids, oligonucleotides, blood components, serum components, lipids, pharmaceutical agents and compounds of low molecular weight, or antibodies and antibody fragments.

4. Device according to claim 1, whereby the change in the emission properties of the portion of the emitter is selected from a change in the polarization plane, the fluorescence intensity, the phosphorescence intensity, the fluorescence service life and a bathochromic shift of the absorption maximum and/or the fluorescence maximum.

5. Device according to claim 1, whereby the substance-detecting agents are peptides, proteins, oligonucleotides and especially antibodies or antibody fragments.

6. Device according to claim 3, whereby the antibodies or antibody fragments are selected from polyclonal or monoclonal antibodies, humanized antibodies, Fab fragments, especially monomeric Fab fragments, scFv fragments, synthetic and recombinant antibodies, scTCR chains and mixtures thereof.

7. Device according to claim 5, whereby the anti-substance antibody or the anti-substance antibody fragment exhibits a higher antigen-binding affinity for the emitter than the anti-emitter antibody or the anti-emitter antibody fragment.

8. Device according to claim 7, whereby the anti-substance antibody or the anti-substance-antibody fragment exhibits an at least 2× higher antigen binding affinity for the emitter than the anti-emitter antibody or the anti-emitter antibody fragment.

9. Device according to claim 7, whereby the anti-substance antibody or the anti-substance-antibody fragment exhibits an at least 10× higher antigen binding affinity for the emitter than the anti-emitter antibody or the anti-emitter-antibody fragment.

10. Device according to claim 7, whereby the binding affinity of the anti-substance antibody or the antagonistic-substance-antibody fragment is less than 50 nm and preferably less than 10 nm.

11. Device according to claim 1, whereby the anti-emitter antibody that is immobilized on a surface is present in molar excess compared to the anti-substance antibody or compared to the antigen, whereby the ratio is preferably from 1:2 the anti-substance antibody or compared to the antigen, whereby the ratio is preferably from 1:2 to 1:50.

12. Device according to claim 1, whereby the emitter comprises a dye that exhibits at least an absorption maximum and/or fluorescence maximum within the spectral range of 700 to 1000 nm, preferably at least an absorption maximum and fluorescence maximum within the spectral range of 750 to 900 nm.

13. Device according to claim 1, whereby the shift of the absorption maximum and/or fluorescence maximum takes place at higher wavelengths after interaction with the agent for detecting the emitter by a value of greater than 15 nm, preferably greater than 25 nm, and most preferably by approximately 30 nm.

14. Device according to claim 1, whereby the emitter that is used comprises a dye that is selected from the group of polymethine dyes, such as dicarbocyanine, tricarbocyanine, indotricarbocyanine, merocyanine, styryl, squalirium and oxonol dyes and rhodamine dyes, phenoxazine or phenothiazine dyes.

15. Device according to claim 1, whereby the emitter of the substance-emitter conjugate comprises a cyanine dye of general formula (I) 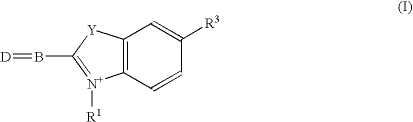
in which D stands for a radical (II) or (III) 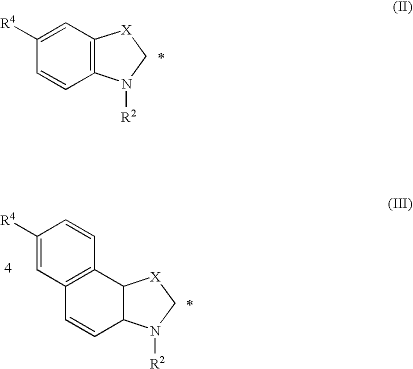
and can stand for the group (IV), (V), (VI), (VII) or (VIII) 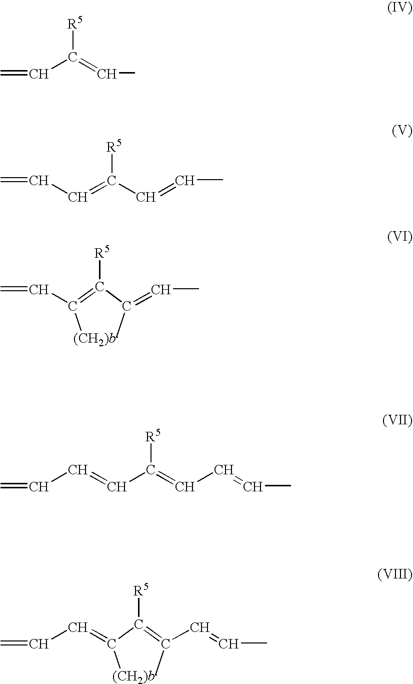
in which R¹ and R², independently of one another, represent a C₁-C₄-sulfoalkyl chain, a saturated or unsaturated, branched or straight-chain C₁-C₅₀-alkyl chain, which optionally is interrupted by 0 to 15 oxygen atoms and/or by 0 to 3 carbonyl groups, and/or can be substituted with 0 to 5 hydroxy groups,
R³ and R⁴, independently of one another, stand for the group —COOE¹, —CONE¹E², —NHCOE¹, —NHCONHE¹, —NE¹E², —OE¹, —OSO₃E¹, —SO₃E¹, —SO₂NHE¹ or —E¹, whereby E¹ and E², independently of one another, represent a hydrogen atom, a C₁-C₄-sulfoalkyl chain, a saturated or unsaturated, branched or straight-chain C₁-C₅₀-alkyl chain, which optionally is interrupted by 0 to 15 oxygen atoms and/or by 0 to 3 carbonyl groups and/or is substituted with 0 to 5 hydroxy groups,
R⁵ stands for a hydrogen atom, a methyl-, ethyl- or propyl radical, or a fluorine, chlorine, bromine or iodine atom,
b means the number 2 or 3, and
X and Y, independently of each other mean O, S, ═C(CH₃)₂ or—(CH═CH)—, and salts and solvates of these compounds. solvates of these compounds.

16. Device according to claim 1, whereby the substance-detecting agent and/or the substance are present directly or indirectly on a surface that is statistically random or is immobilized in a targeted manner.

17. Device according to claim 1, whereby the surface comprises a membrane, ball (bead) or solid flat surface that consists of a resin matrix, silicon, glass, polystyrene, aluminum, steel, iron, copper, nickel, silver or gold.

18. Use of a device according to claim 1 for the in vitro diagnosis.

19. Process for direct quantitative in vitro determination of a substance that is contained in a sample, comprising the steps of
a) making available a device that comprises
i) agents immobilized on a surface for detecting substances,
ii) a free substance-emitter conjugate, and
iii) agents that are immobilized on a surface for detecting the emitter, whereby the emitter that is used comprises a portion that reacts with a change in the emission properties in an interaction with the agents for detecting the emitter,
b) bringing into contact the device with a sample that contains a substance that is to be quantified, and
c) measuring the change in the emission properties of the emitter.

20. Process for direct quantitative in vitro determination of a substance that is contained in a sample, comprising the steps of
b) bringing into contact the device with a sample that contains a substance that is to be quantified, and
c) measuring the change in the emission properties of the emitter.

21. Process for quantitative in vitro determination of a substance that is contained in a sample according to claim 19, further comprising
d) quantification of the substance that is contained in the sample by means of the measured change in the emission properties of the emitter.

22. Process according to claim 19, whereby the substance is selected from antigens, such as proteins, peptides, nucleic acids, oligonucleotides, blood components, serum components, lipids, pharmaceutical agents and compounds of low molecular weight, or antibodies and antibody fragments.

23. Process according to claim 19, whereby the change in the emission properties of the portion of the emitter is selected from a change in the polarization plane, the fluorescence intensity, the phosphorescence intensity, the fluorescence service life and a bathochromic shift of the absorption maximum and/or the fluorescence maximum.

24. Process according to claim 19, whereby as the substance-detecting agents, peptides, proteins, and especially antibodies or antibody fragments are brought into contact with the sample.

25. Process according to claim 1, whereby the antibodies or antibody fragments are selected from polyclonal or monoclonal antibodies, humanized antibodies, Fab fragments, especially monomeric Fab fragments, scFv-fragments, synthetic and recombinant antibodies, scTCR chains and mixtures thereof.

26. Process according to claim 24, whereby the anti-substance antibody or the anti-substance antibody fragment exhibits a higher antigen binding affinity for the emitter than the anti-emitter antibody or the anti-emitter-antibody fragment.

27. Process according to claim 26, whereby the anti-substance antibody or the anti-substance-antibody fragment exhibits an at least 2× higher and especially 10× higher antigen binding affinity for the emitter than the anti-emitter-antibody or the anti-emitter-antibody fragment.

28. Process according to claim 24, whereby the anti-emitter antibody that is immobilized on a surface is brought into contact with the sample in molar excess compared to the anti-substance-antibody or compared to the antigen, whereby the ratio is preferably 1:2-1:50.

29. Process according to claim 24, whereby the emitter comprises a dye that exhibits at least an absorption maximum and/or fluorescence maximum within the spectral range of 700 to 1000 nm, preferably at least an absorption maximum and fluorescence maximum within the spectral range of 750 to 900 nm.

30. Process according to claim 24, whereby the shift of the absorption maximum and/or fluorescence maximum takes place at higher wavelengths after interaction with the agent for detecting the emitter by a value of greater than 15 nm, preferably greater than 25 nm, and most preferably by approximately 30 nm.

31. Process according to claim 24, whereby the emitter that is used comprises a dye that is selected from the group of polymethine dyes, such as dicarbocyanine, tricarbocyanine, indotricarbocyanine, merocyanine, styryl, squarilium and oxonol dyes and rhodamine dyes, phenoxazine or phenothiazine dyes.

32. Process according to claim 24, whereby the emitter of the substance-emitter conjugate comprises a cyanine dye of general formula (I) 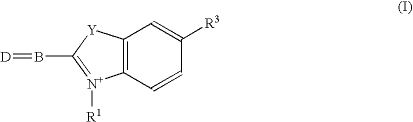
in which D stands for a radical (II) or (III) 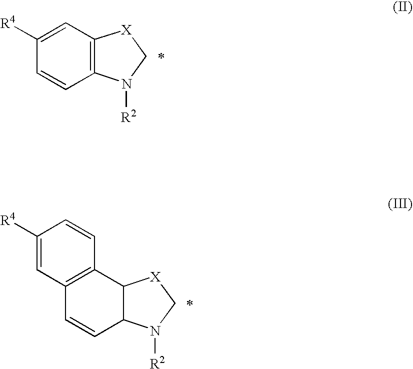
whereby the position that is labeled with the star means the point of linkage with radical B
and can stand for the group (IV), (V), (VI), (VII) or (VIII) 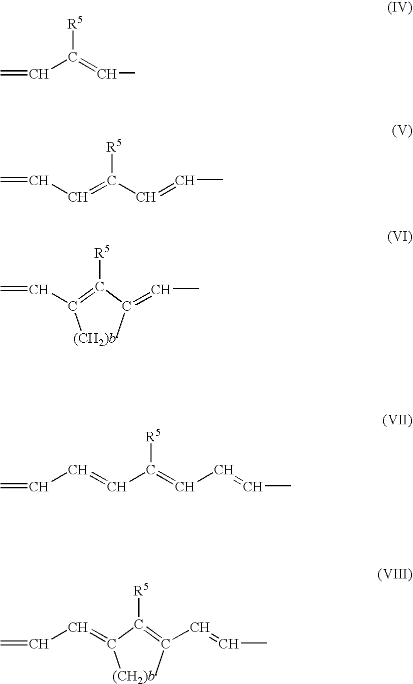
in which R¹ and R², independently of one another, represent a C₁-C₄-sulfoalkyl chain, a saturated or unsaturated, branched or straight-chain C₁-C₅₀-alkyl chain, which optionally is interrupted by 0 to 15 oxygen atoms and/or by 0 to 3 carbonyl groups and/or can be substituted with 0 to 5 hydroxy groups;
R³ and R⁴, independently of one another, stand for the group —COOE¹, —CONE¹E², —NHCOE¹, —NHCONHE¹, —NE¹E², —OE¹, —OSO₃E¹, —SO₃E¹, —SO₂NHE¹ or —E¹, whereby E¹ and E², independently of one another, represent a hydrogen atom, a C₁-C₄-sulfoalkyl chain, a saturated or unsaturated, branched or straight-chain C₁-C₅₀-alkyl chain, which optionally is interrupted by 0 to 15 oxygen atoms and/or by 0 to 3 carbonyl groups and/or is substituted with 0 to 5 hydroxy groups,
R⁵ stands for a hydrogen atom, a methyl, ethyl or propyl group or a fluorine, chlorine, bromine or iodine atom,
b means the number 2 or 3, and
X and Y independently of each other stand for O, S, ═C(CH₃)₂ or—(CH═CH)—, and salts and solvates of these compounds.

33. Use of the substance-emitter conjugates in a process for direct quantitative in vitro determination according to claim 19.

34. Diagnostic kit that comprises agents for implementing the process according to claim 19, optionally together with other adjuvants and/or instructions, together or in separate containers.


SEQUENCE LISTING (12 sequences)

SEQ ID NO: 1 - protein, 245 aa, artificial
  Fab fragment of variable heavy chain
QVQLVQSGAEVKKPGSSVKVSCKASGGTFNSYAISWVRQAPGQGLEWMGGIHPYFGTANYAQKFQGRVTITADESTSTAYMELSSLRSEDTAVYYCARRWRYRWGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFEQKLISEEDLNGAPWSHPQFEK

SEQ ID NO: 2 - protein, 214 aa, artificial
  Fab fragment of variable light chain
DIVLTQPPSVSGAPGQRVTISCSGSSSNIGSYYVYWYQQLPGTAPKLLIYGNSQRPSGVPDRFSGSKSGTSASLAITGLQSEDEADYYCSSWDSSFSWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 3 - DNA, 741 bp, artificial
  coding for Fab fragment of variable heavy chain
caggtgcaattggttcagtctggcgcggaagtgaaaaaaccgggcagcagcgtgaaagtgagctgcaaagcctccggaggcacttttaattcttatgctatttcttgggtgcgccaagcccctgggcagggtctcgagtggatgggcggtatccatccgtattttggcactgcgaattacgcgcagaagtttcagggccgggtgaccattaccgcggatgaaagcaccagcaccgcgtatatggaactgagcagcctgcgtagcgaagatacggccgtgtattattgcgcgcgtcgttggcgttatcgttggggttttgatatttggggccaaggcaccctggtgacggttagctcagcgtcgaccaaaggtccaagcgtgtttccgctggctccgagcagcaaaagcaccagcggcggcacggctgccctgggctgcctggttaaagattatttcccggaaccagtcaccgtgagctggaacagcggggcgctgaccagcggcgtgcatacctttccggcggtgctgcaaagcagcggcctgtatagcctgagcagcgttgtgaccgtgccgagcagcagcttaggcactcagacctatatttgcaacgtgaaccataaaccgagcaacaccaaagtggataaaaaagtggaaccgaaaagcgaattcgagcagaagctgatctctgaggaggatctgaacggcgcgccgtggagccacccgcagtttgaaaaatgataa

SEQ ID NO: 4 - DNA, 648 bp, artificial
  coding for Fab fragment of variable light chain
gatatcgtgctgacccagccgccttcagtgagtggcgcaccaggtcagcgtgtgaccatctcgtgtagcggcagcagcagcaacattggttcttattatgtgtattggtaccagcagttgcccgggacggcgccgaaacttctgatttatggtaattctcagcgtccctcaggcgtgccggatcgttttagcggatccaaaagcggcaccagcgcgagccttgcgattacgggcctgcaaagcgaagacgaagcggattattattgctcttcttgggattcttctttttcttgggtgtttggcggcggcacgaagttaaccgttcttggccagccgaaagccgcaccgagtgtgacgctgtttccgccgagcagcgaagaattgcaggcgaacaaagcgaccctggtgtgcctgattagcgacttttatccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaggggagcaccgtggaaaaaaccgttgcgccgactgaggcctgataa

SEQ ID NO: 5 - protein, 252 aa, artificial
  Fab fragment of variable heavy chain
QVQLQQSGPGLVKPSQTLSLTCAISGDSVSSNSAAWSWIRQSPGRGLEWLGRIYYRSKWYNDYAVSVKSRITINPDTSKNQFSLQLNSVTPEDTAVYYCARTSFYQKLFFIAFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFEQKLISEEDLNGAPWSHPQFEK

SEQ ID NO: 6 - protein, 216 aa, artificial
  Fab fragment of variable light chain
DIALTQPASVSGSPGQSITISCTGTSSDGGAYHYVSWYQQHPGKAPKLMIYGVIYRPSGVSNRFSGSKSGNTASLTISGLQAEDEADYYCAAWDFRKRLNVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 7 - DNA, 762 bp, artificial
  coding for Fab fragment of variable heavy chain
caggtgcaattgcaacagtctggtccgggcctggtgaaaccgagccaaaccctgagcctgacctgtgcgatttccggagatagcgtgagctctaattctgctgcttggtcttggattcgccagtctcctgggcgtggcctcgagtggctgggccgtatctattatcgtagcaagtggtataacgattatgcggtgagcgtgaaaagccggattaccatcaacccggatacttcgaaaaaccagtttagcctgcaactgaacagcgtgaccccggaagatacggccgtgtattattgcgcgcgtacttctttttatcagaagcttttttttattgcttttgatcattggggccaaggcaccctggtgacggttagctcagcgtcgaccaaaggtccaagcgtgtttccgctggctccgagcagcaaaagcaccagcggcggcacggctgccctgggctgcctggttaaagattatttcccggaaccagtcaccgtgagctggaacagcggggcgctgaccagcggcgtgcatacctttccggcggtgctgcaaagcagcggcctgtatagcctgagcagcgttgtgaccgtgccgagcagcagcttaggcactcagacctatatttgcaacgtgaaccataaaccgagcaacaccaaagtggataaaaaagtggaaccgaaaagcgaattcgagcagaagctgatctctgaggaggatctgaacggcgcgccgtggagccacccgcagtttgaaaaatgataa

SEQ ID NO: 8 - DNA, 654 bp, artificial
  coding for Fab fragment of variable light chain
gatatcgcactgacccagccagcttcagtgagcggctcaccaggtcagagcattaccatctcgtgtacgggtactagcagcgatggtggtgcttatcattatgtgtcttggtaccagcagcatcccgggaaggcgccgaaacttatgatttatggtgttatttatcgtccctcaggcgtgagcaaccgttttagcggatccaaaagcggcaacaccgcgagcctgaccattagcggcctgcaagcggaagacgaagcggattattattgcgctgcttgggattttcgtaagcgtcttaatgtgtttggcggcggcacgaagttaaccgttcttggccagccgaaagccgcaccgagtgtgacgctgtttccgccgagcagcgaagaattgcaggcgaacaaagcgaccctggtgtgcctgattagcgacttttatccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaggggagcaccgtggaaaaaaccgttgcgccgactgaggcctgataa

SEQ ID NO: 9 - protein, 246 aa, artificial
  Fab fragment of variable heavy chain
QVQLVQSGAEVKKPGSSVKVSCKASGGTFSNYAINWVRQAPGQGLEWMGNIEPYFGTANYAQKFQGRVTITADESTSTAYMELSSLRSEDTAVYYCARYFMSYKHLSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFEQKLISEEDLNGAPWSHPQFEK

SEQ ID NO: 10 - protein, 216 aa, artificial
  Fab fragment of variable light chain
DIALTQPASVSGSPGQSITISCTGTSSDVGSNNYVSWYQQHPGKAPKLMIYGGSNRPSGVSNRFSGSKSGNTASLTISGLQAEDEADYYCQSWTTRPLNRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 11 - DNA, 744 bp, artificial
  coding for Fab fragment of variable heavy chain
caggtgcaattggttcagtctggcgcggaagtgaaaaaaccgggcagcagcgtgaaagtgagctgcaaagcctccggaggcactttttctaattatgctattaattgggtgcgccaagcccctgggcagggtctcgagtggatgggcaatatcgagccgtattttggcactgcgaattacgcgcagaagtttcagggccgggtgaccattaccgcggatgaaagcaccagcaccgcgtatatggaactgagcagcctgcgtagcgaagatacggccgtgtattattgcgcgcgttattttatgtcttataagcatctttctgattattggggccaaggcaccctggtgacggttagctcagcgtcgaccaaaggtccaagcgtgtttccgctggctccgagcagcaaaagcaccagcggcggcacggctgccctgggctgcctggttaaagattatttcccggaaccagtcaccgtgagctggaacagcggggcgctgaccagcggcgtgcatacctttccggcggtgctgcaaagcagcggcctgtatagcctgagcagcgttgtgaccgtgccgagcagcagcttaggcactcagacctatatttgcaacgtgaaccataaaccgagcaacaccaaagtggataaaaaagtggaaccgaaaagcgaattcgagcagaagctgatctctgaggaggatctgaacggcgcgccgtggagccacccgcagtttgaaaaatgataa

SEQ ID NO: 12 - DNA, 654 bp, artificial
  coding for Fab fragment of variable light chain
gatatcgcactgacccagccagcttcagtgagcggctcaccaggtcagagcattaccatctcgtgtacgggtactagcagcgatgttggttctaataattatgtgtcttggtaccagcagcatcccgggaaggcgccgaaacttatgatttatggtggttctaatcgtccctcaggcgtgagcaaccgttttagcggatccaaaagcggcaacaccgcgagcctgaccattagcggcctgcaagcggaagacgaagcggattattattgccagtcttggactactcgtcctcttaatcgtgtgtttggcggcggcacgaagttaaccgttcttggccagccgaaagccgcaccgagtgtgacgctgtttccgccgagcagcgaagaattgcaggcgaacaaagcgaccctggtgtgcctgattagcgacttttatccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaggggagcaccgtggaaaaaaccgttgcgccgactgaggcctgataa